(19) United States
(12) Patent Application Publication
(10) Pub. No.: US 2024/0043438 A1
(43) Pub. Date: Feb. 8, 2024

(54) METHODS FOR MAKING DARUNAVIR P2-LIGAND PRECURSORS

(71) Applicants: Arun K. Ghosh, West Lafayette, IN (US); William Lewis Robinson, Taft, TN (US); Shivaji Markad, West Lafayette, IN (US)

(72) Inventors: Arun K. Ghosh, West Lafayette, IN (US); William Lewis Robinson, Taft, TN (US); Shivaji Markad, West Lafayette, IN (US)

(21) Appl. No.: 18/041,072

(22) PCT Filed: Jun. 11, 2021
(86) PCT No.: PCT/US2021/037117

§ 371 Date: Feb. 8, 2023

Related U.S. Application Data
(60) Provisional application No. 63/064,279, filed on Aug. 11, 2020.

(51) Int. Cl. C07D 493/04

(52) U.S. Cl. CPC C07D 493/04

(57) ABSTRACT

A method for making an optically active P2-ligand precursor comprising converting D-xylose or a derivative thereof or D-glucose or a derivative thereof to the optically active P2-ligand precursor.

### CROSS REFERENCE TO RELATED APPLICATION

This application claims priority to U.S. provisional patent application No. 63/064,279, which was filed on Aug. 11, 2020, and which is hereby incorporated by reference in its entirety.

### STATEMENT OF U.S. GOVERNMENT SUPPORT

This invention was made with government support under AI150466 awarded by the National Institutes of Health. The government has certain rights in the invention.

### BACKGROUND

Darunavir is the most recent FDA-approved HIV-1 protease inhibitor drug for the treatment of patients with HIV-1 infection and AIDS. It is exceedingly potent and has exhibited broad-spectrum activity against highly multidrug-resistant HIV-1 variants. It received FDA approval in 2006 for the treatment of HIV/AIDS patients who are harboring multidrug-resistant HIV-1 variants and who do not respond to other approved therapies. Darunavir received full approval in 2008 for all HIV/AIDS patients including pediatrics.

Darunavir is a widely used protease inhibitor drug and it has become the front-line therapy for treatment of HIV/AIDS. Darunavir has been specifically designed to promote extensive hydrogen bonding interactions with HIV-1 protease active site backbone atoms. One of the key features of darunavir is the stereochemically defined (3R,3aS,6aR)-bis-tetrahydrofuran (bis-THF) heterocycles as the P2-ligand. Extensive structure-activity studies and X-ray crystallographic studies have established the bis-THF ligand as the privileged ligand for S2 subsite of HIV-1 protease for a variety of very potent HIV-1 protease inhibitors with clinical potential. The bis-THF ligand contains three contiguous stereo centers. A number of syntheses of bis-THF ligand have been reported in the literature. But those syntheses tend to be lengthy, complicated, and/or use expensive starting materials. Accordingly, there is a need for synthetic schemes yielding, e.g., P2-ligand precursors (e.g., a P2-ligand alcohol such as compound 9 described herein in FIGS. 1 and 2) that are shorter, simplified, and/or use cheaper starting materials.

The above and other objects, features, and advantages of the present disclosure will become more apparent from the detailed description and figures.

### SUMMARY

The disclosure relates to, among other things, an optically active synthesis of a P2-ligand precursor for the darunavir bis-THF P2 ligand utilizing inexpensive D-xylose or D-glucose as the starting material or commercially available derivatives, such as compounds 2 and 11, described herein and having the formulae:

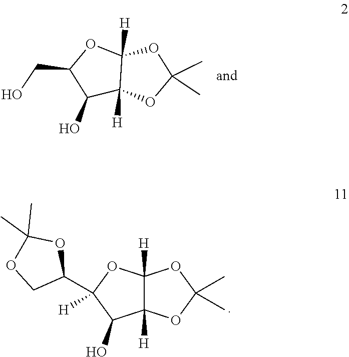

### BRIEF DESCRIPTION OF THE DRAWINGS

FIG. 1 is a synthetic scheme for an optically active P2-ligand precursor for darunavir from D-xylose or commercially available derivative 2.

FIG. 2 is a synthetic scheme for an optically active P2-ligand precursor for darunavir from D-glucose or commercially available derivative 11.

It is to be understood that the drawings are not intended to limit the scope of the present teachings in any way.

### DETAILED DESCRIPTION

In the broadest sense, the disclosure relates to a method for making an optically active P2-ligand precursor comprising converting D-xylose or a derivative thereof or D-glucose or a derivative thereof to the optically active P2-ligand precursor. For example, the disclosure relates to a method for making an optically active P2-ligand precursor, alcohol 9, from D-xylose or a derivative thereof or D-glucose or a derivative thereof. An example of the methods contemplated herein is shown in Scheme 1:

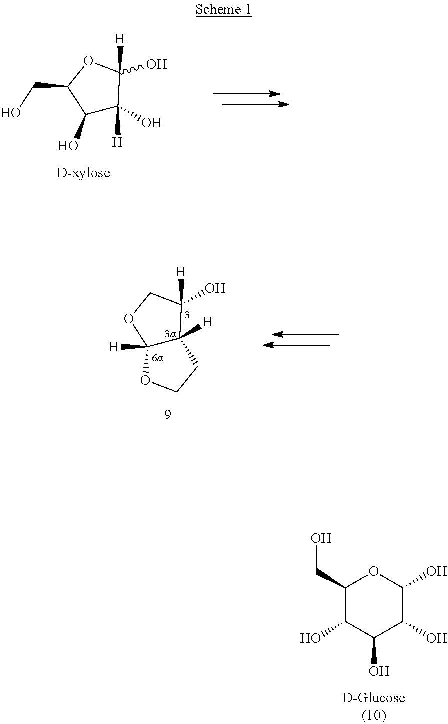

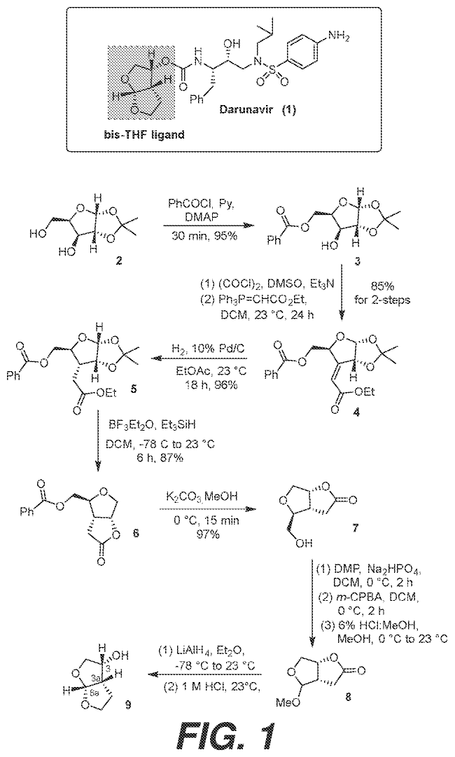

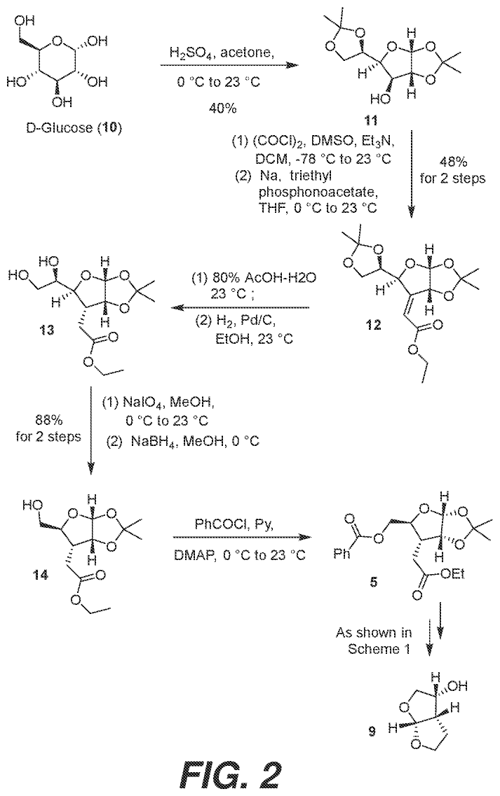

An example of a D-xylose derivative is compound 2 in FIG. 1. And an example of a D-glucose derivative is compound 11 in FIG. 2.

More specifically, the disclosure relates to a method for making an optically active P2-ligand precursor, alcohol 9, from D-xylose or D-glucose, via intermediate ester 5 as shown in Scheme 2:

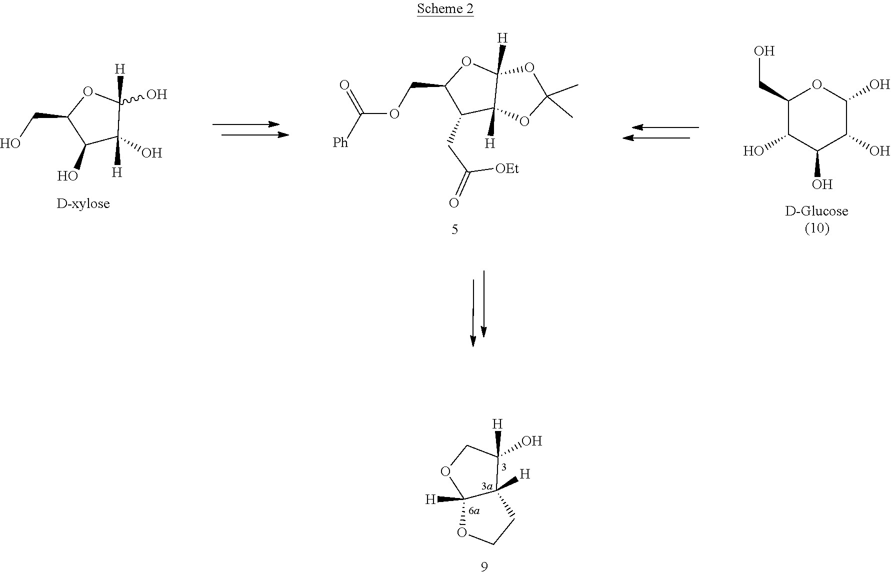

One specific approach for making an optically active P2-ligand precursor for darunavir is shown in FIG. 1 and begins with acetonide-protected xylofuranose 2, which is derived from D-xylose and is commercially available. The protected xylofuranose 2 is treated with benzoyl chloride to give the benzyl-protected compound 3, which is subsequently oxidized under Swern-conditions (e.g., in dimethyl sulfoxide (DMSO) with oxalyl chloride). The resulting compound was reacted with a Wittig reagent to give compound 4. The exocyclic double bond of compound 4 is then stereoselectively reduced to give compound 5. The acetonide protecting group is removed using suitable conditions (e.g., BF₃ etherate in the presence of triethylsilane) to give compound 6 and the resulting acetal was reduced. The benzoyl protecting group is subsequently removed under suitable conditions to give compound 7. Compound 7 is converted to compound 8 by first oxidizing the exocyclic hydroxymethyl group into the corresponding aldehyde:

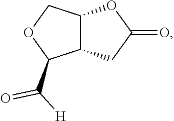 [SMILES: O=C[C@H]1OC[C@@H]2OC(=O)C[C@@H]21]

which is, in turn, converted to the corresponding formate (e.g., via Baeyer-Villiger oxidation):

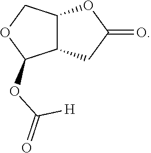 [SMILES: O=CO[C@H]1OC[C@@H]2OC(=O)C[C@H]12]

The formate is subsequently transformed into the corresponding alkoxide (e.g., methoxide) as shown in FIG. 1:

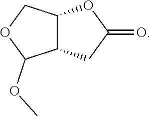

The lactone is converted to the P2-ligand precursor, alcohol 9, under the conditions shown in FIG. 1.

Another specific approach for making an optically active P2-ligand precursor for Darunavir is shown in FIG. 2 and begins with D-glucose 10, which is converted to bis-acetonide 11, which is commercially available. Bis-acetonide 11 is oxidized under Swern-conditions (e.g., in DMSO with oxalyl chloride). The resulting compound was reacted with a Wittig reagent (e.g., triethyl phosphonoacetate) to give compound 12. The exocyclic acetonide group was removed and the resulting double bond of compound 12 is then stereoselectively reduced to give compound 13. Periodate oxidation to give the corresponding aldehyde, and subsequent reduction (e.g., using NaBH₄) gives alcohol 14. Treatment of alcohol 14 with benzoyl chloride gives compound 5. As shown in FIG. 1, the acetonide protecting group of compound 5 is removed and the resulting acetal is reduced using suitable conditions (e.g., BF₃ etherate in the presence of triethylsilane) to give compound 6. The benzoyl protecting group is subsequently removed under suitable conditions to give compound 7. Compound 7 is converted to compound 8 by first oxidizing the exocyclic hydroxymethyl group into the corresponding aldehyde:

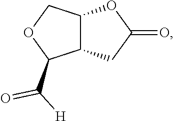 [SMILES: O=C[C@H]1OC[C@@H]2OC(=O)C[C@@H]21]

which is, in turn, converted to the corresponding formate (e.g., via Baeyer-Villiger oxidation):

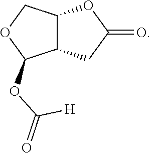 [SMILES: O=CO[C@H]1OC[C@@H]2OC(=O)C[C@H]12]

The formate is subsequently transformed into the corresponding alkoxide (e.g., methoxide) as shown in FIG. 1:

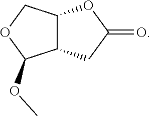

The lactone is converted to the P2-ligand precursor, alcohol 9, under the conditions shown in FIG. 1.

Although the synthetic schemes shown in FIGS. 1 and 2 show specific reaction sequences and specific intermediates, it is contemplated that there may be other reaction sequences not specified herein that can be used by those of skill in the art to access the P2-ligand precursor, alcohol 9, via D-xylose or D-glucose. And those reaction sequences can proceed through compound 5 or any other suitable intermediate.

The term “alkoxy” as used herein refers to an oxygen atom connected to an alkyl group, including a cycloalkyl group, as defined herein. Examples of linear alkoxy groups include, but are not limited to, methoxy, ethoxy, propoxy, butoxy, pentyloxy, hexyloxy, and the like. Examples of branched alkoxy include, but are not limited to, isopropoxy, sec-butoxy, tert-butoxy, isopentyloxy, isohexyloxy, and the like. Examples of cyclic alkoxy include, but are not limited to, cyclopropyloxy, cyclobutyloxy, cyclopentyloxy, cyclohexyloxy, and the like. An alkoxy group can include one to about 12-20 or about 12-40 carbon atoms bonded to the oxygen atom, and can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “alkyl” as used herein refers to substituted or unsubstituted straight-chain and branched alkyl groups and cycloalkyl groups having from 1 to 40 carbon atoms (C₁-C₄₀), 1 to about 20 carbon atoms (C₁-C₂₀), 1 to 12 carbons (C₁-C₁₂), 1 to 8 carbon atoms (C₁-C₈), or, in some embodiments, from 1 to 6 carbon atoms (C₁-C₆). Examples of straight-chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and ante-isoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halo groups.

The term “amino” as used herein refers to a substituent of the form —NH₂, —NHR, —NR₂, —NR₃⁺, wherein each R is defined herein, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “akylamino” group includes a monoakylamino, dialkylamino, and trialkylamino group.

The terms “halo,” “halogen,” and “halide”, as used herein, by themselves or as part of another substituent, mean, unless otherwise stated, a fluorine, chlorine, bromine, or iodine atom.

The term “enantiomerically pure” refers to the enantiomeric purity of one or more stereocenter (e.g. all stereocenters) of a compound, wherein the enantiomeric excess (“e.e”) is at least 97%, 98%, 99% or 100%.

### EXAMPLES

The present disclosure can be better understood by reference to the following examples which are offered by way of illustration. The present invention is not limited to the examples given herein.

General Methods. All reactions were carried out under an atmosphere of argon in oven-dried (120° C.) glassware with magnetic stirring unless otherwise noted. Solvents, reagents and chemicals were purchased from commercial suppliers. Solvents were purified as follows: CH₂Cl₂ was distilled from calcium hydride or purified using a solvent purification system; methanol was used without further purification; tetrahydrofuran was distilled from sodium/benzophenone. Purification of reaction products was carried out by flash chromatography using either silica gel 230-400 mesh (60 Å pore diameter) or alumina 80-200 mesh. Analytical thin layer chromatography (TLC) was performed on glass-backed silica gel TLC plates (0.25 mm thickness, 60 Å, F-254 indicator) or alumina TLC plates (0.25 mm thickness, UV254). Optical rotations were measured by using a digital polarimeter with a sodium lamp. ¹H Nuclear Magnetic Resonance (NMR) spectra were recorded at 23° C. on a 400 MHz spectrometer and are reported in ppm relative to solvent signals (CDCl₃ at δ=7.26 ppm) as an internal standard. Data are reported as (s=singlet, d=doublet, t=triplet, q=quartet, m=multiplet, dd=doublet of doublets, ddd=doublet of doublet of doublets, dddd=doublet of doublet of doublets of doublets, td=triplet of doublets, qd=quartet of doublets, dt=doublet of triplets, dq=doublet of quartets, brs=broad singlet; coupling constant(s) in Hz; integration). Proton-decoupled ¹³C NMR spectra were recorded on a 100 MHz spectrometer and are reported in ppm by using the solvent as the internal standard (CDCl₃ at δ=77.16 ppm). Low resolution mass spectra were obtained using a Quadrupole LCMS instrument under ESI+. High resolution mass spectra were obtained by the Mass Spectrometry Center at Purdue University. These experiments were performed under ESI+ and APCI+ conditions using an Orbitrap XL instrument.

### Example 1

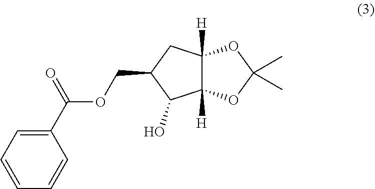 [SMILES: CC1(C)O[C@@H]2[C@H](O)[C@@H](COC(=O)c3ccccc3)C[C@@H]2O1]

((3aR,5R,6aR)-6-Hydroxy-2,2-dimethyltetrahydrofuro[2,3-d][1,3]dioxol-5-yl)methyl benzoate (3). A solution of commercially available 1,2-O-Isopropylidene-α-D-xylofuranose 2 (3.00 g, 15.77 mmol) in dry dichloromethane (DCM) (40 mL) was cooled to 0° C. and to it were added 1.90 mL (23.65 mmol) pyridine and a catalytic amount (192 mg) of N,N-dimethylaminopyridine. The resulting mixture was stirred at 0° C. for 10 min, at which time 2.00 mL (17.34 mmol) benzoyl chloride were added to it dropwise over a period of 30 min. The reaction mixture was stirred at 0° C. for an additional 30 min and then quenched by the addition of 20 mL of a saturated solution of NH₄Cl. The reaction was allowed to warm to room temperature, the layers were separated and the aqueous layer was extracted with 3×10 mL of DCM. The combined organic extracts were washed with 3×10 mL of aqueous solution of CuSO₄, 2×10 mL of water and brine. The organic solution was dried over anhydrous NaSO₄, filtered and concentrated. The crude product was purified by silica gel column chromatography (50% ethyl acetate (EtOAc) in hexane) to afford 3 (4.43 g, 95%). [α]_{D}²³=+15.5 (c=0.15, CHCl₃). ¹H NMR (400 MHz, CDCl₃) δ 8.08-8.01 (m, 2H), 7.62-7.54 (m, 1H), 7.49-7.40 (m, 2H), 5.95 (d, J=3.6 Hz, 1H), 4.83-4.73 (m, 1H), 4.59 (d, J=3.6 Hz, 1H), 4.43-4.34 (m, 2H), 4.18 (dd, J=4.2, 2.3 Hz, 1H), 3.36-3.31 (m, 1H), 1.50 (s, 3H), 1.32 (s, 3H). ¹³C NMR (100 MHz, CDCl₃) δ 167.3, 133.5, 129.8 (2C), 129.1, 128.4 (2C), 111.8, 104.6, 84.9, 78.4, 74.3, 61.2, 26.7, 26.0. LRMS (ESI) m/z: [M+H]⁺ 295.1. HRMS (ESI) m/z: [M+Na]⁺ calcd C₁₅H₁₈O₆Na 317.0996; found 317.0998.

### Example 2

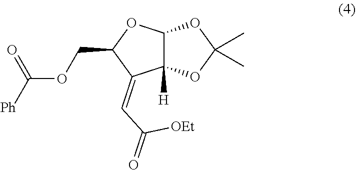 [SMILES: CCOC(=O)/C=C1\[C@H]2OC(C)(C)O[C@H]2O[C@@H]1COC(=O)c1ccccc1]

(3aR,5R,6aS)-2,2-Dimethyl-6-oxotetrahydrofuro[2,3-d][1,3]dioxol-5-yl)methyl benzoate (4). A solution of oxalyl chloride (2.42 mL, 28.54 mmol) in 40 mL of anhydrous DCM was cooled to −78° C. under an argon atmosphere. To this DMSO (4.04 mL, 57.08 mmol) was added dropwise over a period of 15 min. After the resulting solution had been stirred at the same temperature for 10 min, a solution of alcohol 3 (4.2 g, 14.27 mmol) in anhydrous DCM (10 mL) was added to it dropwise over a period of 15 min. Stirring was continued at −78° C. for an additional 30 min. Then, triethylamine (Et₃N) (9.92 mL, 71.35 mmol) was added. The temperature of the reaction mixture was maintained at −78° C. for 10 min, and the mixture was allowed to stir for 30 min while warming to 23° C. The reaction was then quenched by 30 mL of water and extracted with (3×10 mL) DCM. The DCM layer was washed with saturated aqueous NaHCO₃ and then brine and was dried over anhydrous Na₂SO₄. Filtration and solvent removal afforded crude product which was used in the next step without further purification.

To a stirred solution of the above ketone (14.27 mmol) in dry DCM (30 mL) was added (Carbethoxymethylene)triphenylphophorane (5.96 g, 17.12 mmol). The reaction mixture was stirred under argon at 23° C. for 24 h. The reaction mixture was concentrated under reduced pressure and residue was purified by flash chromatography on silica (5% EtOAc/hexanes to 10% EtOAc/hexane) to yield ester as an 8:1 (Z:E) mixture of separable isomers. Z-isomer of ester 4 (4.4 g, 85% over 2 steps), yellow oil. [α]_{D}²³=+240 (c=0.5, CHCl₃). ¹H NMR (400 MHz, CDCl₃) δ 8.05-7.92 (m, 2H), 7.62-7.53 (m, 1H), 7.44 (dd, J=8.4, 7.2 Hz, 2H), 6.03-5.95 (m, 2H), 5.78 (dt, J=4.2, 1.5 Hz, 1H), 5.16 (ddt, J=5.3, 3.7, 1.8 Hz, 1H), 4.57 (dd, J=11.9, 3.5 Hz, 1H), 4.43 (dd, J=11.9, 5.0 Hz, 1H), 4.24 (q, J=7.1 Hz, 2H), 1.51 (s, 3H), 1.44 (s, 3H), 1.30 (t, J=7.1 Hz, 3H). ¹³C NMR (100 MHz, CDCl₃) δ 166.0, 164.5, 154.5, 133.2, 129.6 (2C), 129.4, 128.4 (2C), 117.1, 112.9, 105.0, 78.1, 77.8, 65.1, 60.8, 27.3, 27.0, 14.0. LRMS (ESI) m/z: [M+Na]⁺ 385.0. HRMS (ESI) m/z: [M+Na]⁺ calcd C₁₉H₂₂O₇Na 385.1258; found 385.1247.

### Example 3

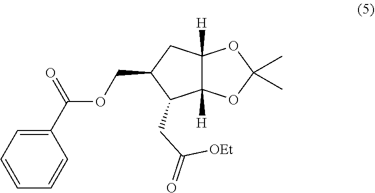 [SMILES: CCOC(=O)C[C@@H]1[C@@H](COC(=O)c2ccccc2)C[C@@H]2OC(C)(C)O[C@H]12]

((3aR,5S,6R,6aR)-6-(2-Ethoxy-2-oxoethyl)-2,2-dimethyltetrahydrofuro[2,3-d][1,3]dioxol-6-yl)methyl benzoate (5). To a flask were added Z-ester 4 (4.2 g, 11.59 mmol) and anhydrous ethanol (70 mL) was added 10% Pd/C (147 mg, 5% w/w). The flask was evacuated by vacuum and flushed with argon three times. Next, the flask was evacuated by vacuum and flushed with hydrogen three times. The reaction was then left to stir under an atmosphere of hydrogen (1 atm) for 6 h. upon completion of the reaction, the mixture was filtered through celite with EtOAc and concentrated to yield colorless oil that was purified by flash chromatography on silica (15% EtOAc/hexane) to yield ester 5 (4.02 g, 96%) as a mixture of inseparable diastereomers 8:1. [α]_{D}²³=+52.7 (c=0.584, CHCl₃). ¹H NMR (400 MHz, CDCl₃) δ 8.15-8.00 (m, 2H), 7.60-7.52 (m, 1H), 7.43 (t, J=7.6 Hz, 2H), 5.87 (d, J=3.7 Hz, 1H), 4.82 (t, J=4.2 Hz, 1H), 4.56 (dd, J=12.3, 2.9 Hz, 1H), 4.34 (dd, J=12.3, 5.0 Hz, 1H), 4.19-4.08 (m, 3H), 2.75 (dd, J=17.0, 9.8 Hz, 1H), 2.54-2.33 (m, 2H), 1.53 (s, 3H), 1.32 (s, 3H), 1.25 (t, J=7.1 Hz, 3H). ¹³C NMR (101 MHz, CDCl₃) δ 171.7, 166.3, 133.1, 129.7 (2C), 129.6, 128.3 (2C), 111.7, 104.8, 80.8, 78.6, 63.9, 60.7, 41.4, 29.6, 26.6, 26.2, 14.0. LRMS (ESI) m/z: [M+Na]⁺ 387.0. HRMS (ESI) m/z: [M+Na]⁺ calcd C₁₉H₂₄O₇Na 387.1414; found 387.1421.

### Example 4

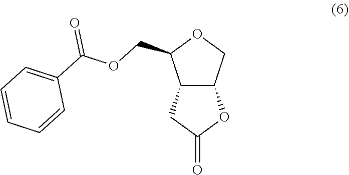 [SMILES: O=C1C[C@H]2[C@H](CO[C@@H]2COC(=O)c2ccccc2)O1]

((3aR,4S,6aR)-2-Oxohexahydrofuro[3,4-b]furan-4-yl)methyl benzoate (6). To a flask was added ester 5 (2.3 g, 6.31 mmol) in dry DCM (20 mL) at −78° C. then BF₃Et₂O (6.18 mL, 50.48 mmol) was added dropwise over 5 min followed by addition of Et₃SiH (3.01 mL, 18.93 mmol). The reaction mixture was stirred at −78° C. to 23° C. for 6 h. The reaction mixture was cooled to 0° C. and quenched with saturated solution of NaHCO₃ (20 mL) and extracted with (3×10 mL) DCM. The combined organic layer was washed with water (20 mL) and brine. The organic solution was dried over anhydrous NaSO₄, filtered and concentrated. The product was purified by silica gel column chromatography (50% EtOAc/hexane) to afford 6 (1.44 gm, 87%). [α]_{D}²³=+9.6 (c=0.015, CHCl₃). ¹H NMR (400 MHz, CDCl₃) δ 8.06-7.97 (m, 2H), 7.65-7.54 (m, 1H), 7.52-7.35 (m, 2H), 5.15 (ddd, J=6.7, 4.8, 1.9 Hz, 1H), 4.53-4.35 (m, 2H), 4.24 (ddd, J=11.2, 4.8, 0.5 Hz, 1H), 4.17-4.04 (m, 2H), 2.99-2.84 (m, 2H), 2.57 (dd, J=17.8, 1.7 Hz, 1H). ¹³C NMR (101 MHz, CDCl₃) δ 175.3, 166.1, 133.3, 129.6 (2C), 129.3, 128.4 (2C), 83.9, 82.9, 72.9, 64.6, 41.4, 33.0. LRMS (ESI) m/z: [M+H]⁺ 263.0. HRMS (ESI) m/z: [M+H]⁺ calcd C₁₄H₁₅O₅ 263.0914; found 263.0920.

### Example 5

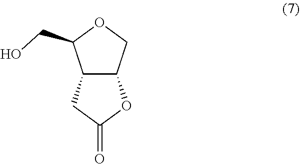 [SMILES: O=C1C[C@H]2[C@H](CO[C@@H]2CO)O1]

(3aR,4S,6aR)-4-(Hydroxymethyl)tetrahydrofuro[3,4-b]furan-2(3H)-one (7). To a stirred solution of the lactone 6 (1.2 g, 4.58 mmol) in methanol (MeOH) (15 mL) at 0° C. was added K₂CO₃ (695 mg, 5.03 mmol) and stirred for 15 min. The reaction mixture was concentrated under reduced pressure to remove MeOH. The obtained residue was dissolved in water (10 mL) and DCM (10 mL) and aqueous layer was extracted with (3×10 mL) 10% MeOH/DCM. The combined organic layer was washed with brine and dried over anhydrous Na₂SO₄. The solvent was evaporated and residue was purified by silica gel column chromatography (10% MeOH/DCM) to afford 7 (690 mg, 96%) as a colorless oil. [α]_{D}²³=−11.1 (c=0.02, CHCl₃). ¹H NMR (400 MHz, CDCl₃) δ 5.09 (ddd, J=6.9, 4.8, 2.0 Hz, 1H), 4.17 (dd, J=11.0, 4.7 Hz, 1H), 4.02 (dt, J=11.4, 2.4 Hz, 1H), 3.86-3.70 (m, 2H), 3.64-3.56 (m, 1H), 2.98-2.88 (m, 1H), 2.82 (ddd, J=18.1, 9.3, 1.4 Hz, 1H), 2.46 (dd, J=18.1, 1.8 Hz, 1H). ¹³C NMR (101 MHz, CDCl₃) δ 175.6, 85.2, 84.3, 72.7, 62.4, 40.0, 32.8. LRMS (ESI) m/z: [M+H]⁺ 159. HRMS (ESI) m/z: [M+Na]⁺ calcd C₇H₁₀O₄Na 181.0471; found 181.0470.

### Example 6

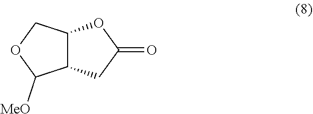 [SMILES: COC1OC[C@@H]2OC(=O)C[C@H]12]

(3aS,6aR)-4-Methoxytetrahydrofuro[3,4-b]furan-2(3H)-one (8). A solution of oxalyl chloride (0.53 mL, 6.3 mmol) in 8 mL of anhydrous DCM was cooled to −78° C. under argon atmosphere. To this, a solution of DMSO (0.89 mL, 12.6 mmol) in 3 mL of DCM was added dropwise over a period of 15 min. After the resulting solution had been stirred at the same temperature for 15 min, a solution of 1 (200 mg, 1.26 mmol) in anhydrous DCM (3 mL) was added to it dropwise over a period of 15 min. Stirring was continued at −78° C. to −50° C. for an additional 3 h. Triethylamine (1.75 mL, 12.6 mol) was then added to the reaction mixture and stirred further for 4 h at −78° C. and then quenched with water (10 mL). The biphasic mixture was allowed to warm to room temperature and the layers were separated. The aqueous layer was extracted with (3×10 mL) DCM. The organic extracts were combined and washed with a saturated solution of NaHCO₃ (10 mL), water (10 mL) and brine. The organic solution was dried over anhydrous NaSO₄, filtered and concentrated to afford aldehyde. The crude product as used in the next step without further purification.

To a stirred solution of the above crude aldehyde (1.26 mmol) in anhydrous DCM (10 mL) at 0° C. were added NaHCO₃ (211 mg, 2.52 mmol) and m-CPBA (577 mg, 2.52 mmol). The reaction mixture was stirred for 2 h at 0° C. and quenched with saturated aqueous solution of NaHCO₃ (5 mL). The aqueous layer was extracted with (3×10 mL) DCM diethyl ether. The combined organic solvent was washed with brine, dried over anhydrous Na₂SO₄, filtered and concentrated to afford product. The crude product was used in the next step without further purification.

To a stirred solution of the above crude formate (1.22 mmol) in MeOH (5 mL) at 0° C. was added slowly 6% HCl/MeOH (5 mL) and stirred for 12 h at 0° C. to 23° C. The reaction mixture was then neutralized with saturated solution of NaHCO₃, concentrated under reduced pressure to remove MeOH, extracted with DCM, and washed with brine. The organic layer was dried over anhydrous Na₂SO₄, filtered, and concentrated under reduced pressure to yield crude solid that was purified by flash chromatography on silica (50% ether/hexane) to yield lactone 8 (82 mg, 41% over 3 steps) as a colorless crystalline solid and as a 3:1 mixture of separable anomers. Major Anomer: [α]_{D}²³=+175 (c=0.02, CHCl₃). ¹H NMR (400 MHz, CDCl₃) δ 5.13 (dd, J=7.1, 3.9 Hz, 1H), 4.87 (s, 1H), 4.09 (d, J=10.9 Hz, 1H), 3.94 (dd, J=10.9, 3.9 Hz, 1H), 3.31 (s, 3H), 3.02 (ddd, J=11.2, 7.1, 4.0 Hz, 1H), 2.83 (dd, J=18.6, 11.3 Hz, 1H), 2.50 (dd, J=18.6, 4.0 Hz, 1H). ¹³C NMR (101 MHz, CDCl₃) δ 175.9, 109.9, 82.9, 70.6, 54.5, 45.0, 31.7. LRMS (ESI) m/z: [M+H]⁺ 159.1. HRMS (ESI) m/z: [M+Na]⁺ calcd C₂₈H₃₇N₃O₈SNa 181.0471; found 181.0472.

### Example 7

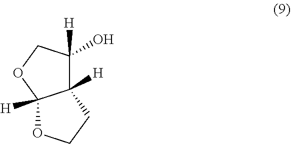

(3R,3aS,6aR)-Hexahydrofuro[2,3-b]furan-3-ol (9). To a flame dried flask was added LiAlH₄ (43.14 mg, 1.13 mmol). The flask was evacuated by vacuum and then flushed with argon. To the flask was added ether (Et₂O) (2 mL). The mixture was stirred and cooled to −78° C. prior to the addition of a solution of lactone 8 (60 mg, 0.37 mmol) in Et₂O (3 ml). The reaction was then allowed to warm to 23° C. over 1 h. The reaction mixture was then cooled to 0° C. and then H₂O (1 mL), 2.0 M NaOH (1 mL) and H₂O (3 mL) were added sequentially and slowly. The mixture was stirred vigorously at 0° C. for 1.5 h and then it was filtered through celite with MeOH. The filtrate was concentrated under reduced pressure to afford a residue containing the crude diol. To the flask containing crude diol (0.37 mmol) was added 1.0 M HCl (10 mL) and the mixture were then extracted with 10% MeOH/DCM. The organic layer was dried over anhydrous Na₂SO₄, filtered and concentrated under reduced pressure to yield the crude ligand alcohol that was purified by flash chromatography on silica (60% ether/hexanes) to yield ligand alcohol 9 (23 mg, 45%) as a colorless oil. [α]_{D}²³=−13.2 (c=0.02, MeOH). ¹H NMR (400 MHz, CDCl₃) δ 5.68 (d, J=5.2 Hz, 1H), 4.44 (q, J=7.1 Hz, 1H), 3.98 (td, J=8.4, 2.1 Hz, 2H), 3.89 (ddd, J=9.9, 8.6, 6.3 Hz, 1H), 3.63 (dd, J=9.2, 7.0 Hz, 1H), 2.85 (dddd, J=10.2, 7.9, 5.1, 2.4 Hz, 1H), 2.30 (ddt, J=12.2, 5.8, 2.6 Hz, 1H), 1.97-1.79 (m, 2H). ¹³C NMR (101 MHz, CDCl₃) δ 109.4, 73.0, 70.9, 69.8, 46.5, 24.7.

### Example 8

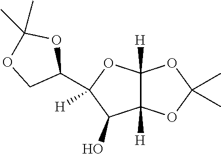

1,2:5,6-Di-O-isopropylidene-α-D-glucofuranose (11). To a 1 L flask was added commercially available α-D-Glucose 10 (15.0 g, 83.3 mmol) and acetone (300 mL, 4085.7 mmol). The mixture was stirred vigorously and cooled to 0° C. prior to dropwise addition of concentrated H₂SO₄ (15.0 mL, 281.4 mmol). The reaction temperature was maintained at 0° C. for 4 h, and then the mixture was allowed to warm to 23° C. over 2 h. The mixture was then cooled to 0° C. and neutralized with 50% aqueous KOH. The mixture was stirred for 12 h while warming to 23° C. The reaction mixture was then filtered through celite with acetone and concentrated under reduced pressure. The residue was extracted with CH₂Cl₂. The organic layer was dried over anhydrous Na₂SO₄, filtered, and concentrated under reduced pressure to yield a crude solid that was recrystallized from boiling hexane, filtered, rinsed with cold acetone, and air-dried to provide diacetone-D-glucose 11 (8.64 g, 40%) as white crystals that were used without further purification. R_{f}=0.50 (50% EtOAc/hexanes, SiO₂). ¹H NMR (400 MHz, CDCl₃) δ 5.94 (d, J=3.6 Hz, 1H), 4.53 (d, J=3.6 Hz, 1H), 4.37-4.31 (m, 2H), 4.17 (dd, J=8.6, 6.2 Hz, 1H), 4.07 (dd, J=7.6, 2.8 Hz, 1H), 3.98 (dd, J=8.6, 5.4 Hz, 1H), 2.58 (d, J=3.2 Hz, 1H), 1.50 (s, 3H), 1.44 (s, 3H), 1.36 (s, 3H), 1.32 (s, 3H). ¹³C NMR (101 MHz, CDCl₃) δ 112.0, 109.8, 105.4, 85.2, 81.2, 75.4, 73.7, 67.8, 27.0, 26.9, 26.3, 25.3.

### Example 9

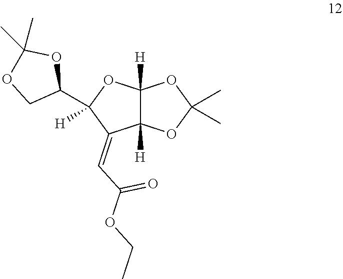

Ethyl 2-((3aR,5S,6aR)-5-((R)-2,2-dimethyl-1,3-dioxolan-4-yl)-2,2-dimethyldihydrofuro[2,3-d][1,3]dioxol-6(5H)-ylidene)acetate (12). To a flame-dried flask was added CH₂Cl₂ (210 mL). The CH₂Cl₂ was stirred under argon and cooled to −78° C. prior to addition of oxalyl chloride (5.47 mL, 64.6 mmol). After 5 min, DMSO (9.18 mL, 129.2 mmol) was added dropwise to the reaction mixture. After 10 min, a solution of commercially available 1,2:5,6-Di-O-isopropylidene-α-D-glucofuranose 11 (8.41 g, 32.3 mmol) in CH₂Cl₂ (20 mL) was added dropwise to the reaction mixture, and then the mixture was stirred at −78° C. for 1 h. At this time, Et₃N (22.52 mL, 161.6 mmol) was added. The temperature of the reaction mixture was maintained at −78° C. for 10 min, and then the cooling bath was removed. The mixture was allowed to stir for 30 min while warming to 23° C. The reaction mixture was then quenched with H₂O (150 mL) and transferred to a separatory funnel. The organic layer was washed with saturated aqueous NaHCO₃ and then with brine. The organic layer was dried over anhydrous Na₂SO₄, filtered, and concentrated under reduced pressure to yield the crude ketone as a brown oil. The crude ketone was used without further purification.

To a flame-dried flask was added NaH (60% in oil) (2.37 g, 59.0 mmol). The flask was evacuated and placed under argon prior to addition of dry THF (91 mL). The mixture was stirred and cooled to 0° C. prior to the dropwise addition of triethyl phosphonoacetate (12.3 mL, 62.2 mmol). After 15 min, a solution of the above crude ketone (31.1 mmol) in dry THF (31 mL) was added slowly at 0° C. The reaction mixture was kept at a temperature of 0° C. for 30 min, and then the reaction mixture was allowed to warm to 23° C. over 30 min. The reaction was quenched by the addition of saturated aqueous NH₄Cl, extracted with Et₂O, and washed with brine. The organic layer was then dried over anhydrous Na₂SO₄, filtered, and concentrated under reduced pressure to yield a brown oil that was purified by flash chromatography on SiO₂ (15% ether/hexanes to 25% ether/hexanes) to yield ester 12 as a 4:1 (Z:E) mixture of separable isomers. Z-isomer of ester 12: (3.95 g, 39% over 2 steps), white solid. R_{f}=0.60 (50% ether/hexanes, SiO₂). ¹H NMR (400 MHz, CDCl₃) δ 6.32 (dd, J=2.2, 1.4 Hz, 1H), 5.82 (d, J=4.2 Hz, 1H), 5.73 (dt, J=4.2, 1.4 Hz, 1H), 4.66 (ddt, J=6.0, 2.2, 1.4 Hz, 1H), 4.28-4.19 (m, 2H), 4.12-4.06 (m, 1H), 4.03-3.97 (m, 2H), 1.49 (s, 3H), 1.43 (s, 3H), 1.39 (s, 3H), 1.35 (s, 3H), 1.30 (t, J=7.1 Hz, 3H). ¹³C NMR (101 MHz, CDCl₃) δ 165.3, 155.8, 118.0, 112.9, 110.3, 105.0, 80.0, 78.5, 76.9, 67.4, 60.8, 27.4, 27.2, 26.8, 25.5, 14.3. E-isomer of ester 12: (963 mg, 9% over 2 steps), colorless oil. R_{f}=0.65 (50% ether/hexanes, SiO₂). ¹H NMR (400 MHz, CDCl₃) δ 6.21 (t, J=1.9 Hz, 1H), 5.92 (d, J=4.8 Hz, 1H), 5.75 (q, J=1.9 Hz, 1H), 5.09 (dt, J=4.8, 1.9 Hz, 1H), 4.34 (ddd, J=7.9, 6.3, 2.6 Hz, 1H), 4.17 (qt, J=7.4, 3.8 Hz, 2H), 3.96 (dd, J=8.8, 6.3 Hz, 1H), 3.56 (dd, J=8.8, 7.9 Hz, 1H), 1.42 (s, 3H), 1.37 (s, 3H), 1.32-1.27 (m, 9H). ¹³C NMR (101 MHz, CDCl₃) δ 165.7, 158.1, 118.2, 113.8, 109.2, 103.9, 82.3, 80.1, 79.1, 65.5, 60.9, 27.99, 27.96, 26.2, 25.8, 14.3.

### Example 10

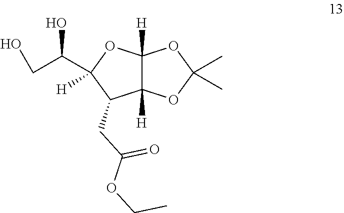 [SMILES: CCOC(=O)C[C@H]1[C@H]2OC(C)(C)O[C@H]2O[C@@H]1[C@H](O)CO]

Ethyl-2-((3aR,5S,6R,6aR)-5-((R)-1,2-dihydroxyethyl)-2,2-dimethyltetrahydrofuro[2,3-d][1,3]dioxol-6-yl)acetate (13). To a flask were added Z-ester 12 (1.77 g, 5.39 mmol) and 80% acetic acid (AcOH)/H₂O (20 mL). The mixture was stirred at 23° C. for 60 h. The reaction mixture was then concentrated, and the resultant crude orange oil was purified by flash chromatography on SiO₂ (50% EtOAc/hexanes) to yield the deprotected α,β-unsaturated ester (1.14 g, 74%) as a colorless oil. R_{f}=0.45 (75% EtOAc/hexanes, SiO₂). ¹H NMR (400 MHz, CDCl₃) δ 6.30 (dd, J=2.2, 1.5 Hz, 1H), 5.86 (d, J=4.2 Hz, 1H), 5.74 (dt, J=4.2, 1.5 Hz, 1H), 4.79 (ddd, J=6.6, 2.2, 1.4 Hz, 1H), 4.23 (q, J=7.1 Hz, 2H), 3.80-3.67 (m, 3H), 2.95 (d, J=6.6 Hz, 1H), 2.45 (s, 1H), 1.48 (s, 3H), 1.41 (s, 3H), 1.30 (t, J=7.1 Hz, 3H). ¹³C NMR (101 MHz, CDCl₃) δ 165.3, 155.7, 117.7, 113.0, 104.9, 80.0, 78.4, 73.5, 63.5, 60.9, 27.4, 27.2, 14.3.

To a flame-dried flask was added a solution of the above α,β-unsaturated ester (1.06 g, 3.68 mmol) in anhydrous ethanol (16 mL) followed by 10% Pd/C (53 mg, 5% w/w). The flask containing the mixture was evacuated by vacuum and flushed with argon three times, and then it was evacuated by vacuum and flushed with hydrogen three times. The reaction mixture was then left to stir under an atmosphere of hydrogen (1 atm) for 24 h. The reaction mixture was then filtered through celite with EtOAc and concentrated under reduced pressure to yield a crude colorless oil that was purified by flash chromatography on SiO₂ (3% MeOH/CH₂Cl₂) to yield the saturated diol 13 (950 mg, 89%) as a colorless syrup. R_{f}=0.45 (75% EtOAc/hexanes, SiO₂). ¹H NMR (400 MHz, CDCl₃) δ 5.76 (d, J=3.7 Hz, 1H), 4.76 (dd, J=4.8, 3.7 Hz, 1H), 4.13 (qd, J=7.1, 3.2 Hz, 2H), 3.82-3.60 (m, 4H), 3.24 (d, J=5.4 Hz, 1H), 2.91 (t, J=5.6 Hz, 1H), 2.77-2.63 (m, 2H), 2.42-2.31 (m, 1H), 1.46 (s, 3H), 1.28 (s, 3H), 1.24 (t, J=7.1 Hz, 3H). ¹³C NMR (101 MHz, CDCl₃) δ 172.9, 111.9, 104.8, 81.6, 81.1, 73.7, 63.9, 60.8, 43.2, 30.5, 26.7, 26.4, 14.3.

### Example 11

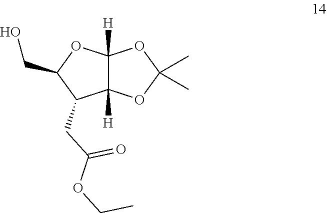 [SMILES: CCOC(=O)C[C@H]1[C@H]2OC(C)(C)O[C@H]2O[C@@H]1CO]

(3aR,4S,6aR 6-Hydroxy-4-(hydroxymethyl)tetrahydrofuro[3,4-b]furan-2(3H)-one (14). To a flask was added saturated diol 13 (866 mg, 2.98 mmol) and MeOH (15 mL). The mixture was stirred and cooled to 0° C. prior to portion-wise addition of NaIO₄ (1.28 g, 5.97 mmol). The reaction mixture was then allowed to warm to 23° C. over 2 h while stirring vigorously. The reaction mixture was filtered through celite with MeOH and concentrated under reduced pressure to yield a residue that was dissolved in H₂O and CH₂Cl₂ and extracted with CH₂Cl₂. The organic layer was dried over anhydrous Na₂SO₄, filtered, and concentrated under reduced pressure to yield the crude aldehyde that was used without further purification. R_{f}=0.80 (75% EtOAc/hexanes, SiO₂).

The above crude aldehyde (2.98 mmol) was dissolved in MeOH (15 mL) and cooled to 0° C. prior to portion-wise addition of NaBH₄ (226 mg, 5.97 mmol). The reaction mixture was stirred at 0° C. for 1 h. The mixture was then concentrated under reduced pressure to remove MeOH. Saturated aqueous NH₄Cl (10 mL) was added, and the organics were extracted with CH₂Cl₂. The organic layer was dried over anhydrous Na₂SO₄, filtered, and concentrated under reduced pressure to yield a colorless oil that was purified by flash chromatography on SiO₂ (40% EtOAc/hexanes) to yield ester 14 (683 mg, 88% over 2 steps) as a colorless oil and as a single diastereomer. Spectral data matched that of the same compound produced by Method A (described above).

### Example 12

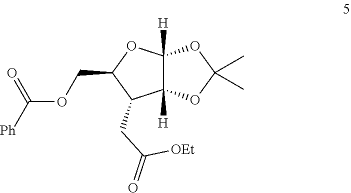 [SMILES: CCOC(=O)C[C@H]1[C@H]2OC(C)(C)O[C@H]2O[C@@H]1COC(=O)c1ccccc1]

((3aR,5S,6R,6aR)-6-(2-Ethoxy-2-oxoethyl)-2,2-dimethyltetrahydrofuro[2,3-d][1,3]dioxol-5-yl)methyl benzoate (5). To a flame dried flask was added a solution of 14 (16 mg, 0.1 mmol) in CH₂Cl₂. To the mixture was added triethylamine (42 μL, 0.3 mmol), and the mixture was then cooled to 0° C. prior to addition of benzoyl chloride (35 μL, 0.3 mmol). The mixture was then allowed to warm to 23° C., and it was stirred under argon for 18 h. The mixture was then diluted with water, extracted with CH₂Cl₂, dried over anhydrous Na₂SO₄, filtered, and concentrated under reduced pressure. Purification by flash chromatography on SiO₂ with 20% EtOAc/hexanes provided the desired benzoate ester 5 (15 mg, 65% yield) as a colorless oil. [α]_{D}²³=+52.7 (c=0.584, CHCl₃). TLC R_{f}=0.30 (20% EtOAc/hexanes, SiO₂ plate). ¹H NMR (400 MHz, CDCl₃) δ 8.15-8.00 (m, 2H), 7.60-7.52 (m, 1H), 7.43 (t, J=7.6 Hz, 2H), 5.87 (d, J=3.7 Hz, 1H), 4.82 (t, J=4.2 Hz, 1H), 4.56 (dd, J=12.3, 2.9 Hz, 1H), 4.34 (dd, J=12.3, 5.0 Hz, 1H), 4.19-4.08 (m, 3H), 2.75 (dd, J=17.0, 9.8 Hz, 1H), 2.54-2.33 (m, 2H), 1.53 (s, 3H), 1.32 (s, 3H), 1.25 (t, J=7.1 Hz, 3H). ¹³C NMR (101 MHz, CDCl₃) δ 171.7, 166.3, 133.1, 129.7 (2C), 129.6, 128.3 (2C), 111.7, 104.8, 80.8, 78.6, 63.9, 60.7, 41.4, 29.6, 26.6, 26.2, 14.0. LRMS (ESI) m/z: [M+Na]⁺ 387.0. HRMS (ESI) m/z: [M+Na]⁺ calcd C₁₉H₂₄O₇Na 387.1414; found 387.1421.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or description that the steps are to be limited to a specific order, it is in no way intended that any particular order be inferred.

The steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

All publications and patents mentioned in the above specification are herein incorporated by reference in their entirety for all purposes. In the event of inconsistent usages between this document and those documents so incorporated by reference, the usage in the incorporated reference should be considered supplementary to that of this document; for irreconcilable inconsistencies, the usage in this document controls.

### Numbered Embodiments

Embodiment 1 relates to a method for making an optically active P2-ligand precursor comprising converting D-xylose or a derivative thereof or D-glucose or a derivative thereof to the optically active P2-ligand precursor.
Embodiment 2 relates to a method of Embodiment 1, wherein the D-xylose derivative is a compound of the formula:

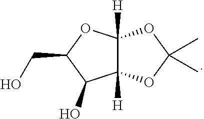

Embodiment 3 relates to a method of Embodiment 1, wherein the D-glucose derivative is a compound of the formula:

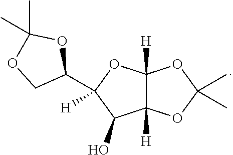

Embodiment 4 relates to a method of any preceding Embodiment, which comprises converting D-xylose or a derivative thereof or D-glucose or a derivative thereof to a compound of the formula:

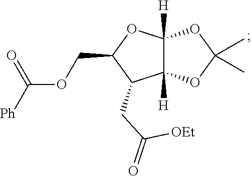 [SMILES: CCOC(=O)C[C@H]1[C@H]2OC(C)(C)O[C@H]2O[C@@H]1COC(=O)c1ccccc1]

; and then
converting said compound to the optically active P2-ligand precursor.
Embodiment 5 relates to a method of Embodiment 4, further comprising converting the compound of the formula:

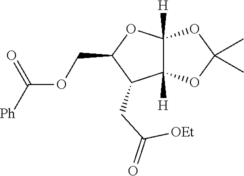 [SMILES: CCOC(=O)C[C@H]1[C@H]2OC(C)(C)O[C@H]2O[C@@H]1COC(=O)c1ccccc1]

to a compound of the formula:

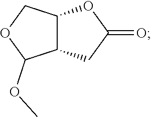

and converting said compound to the optically active P2-ligand precursor.
Embodiment 6 relates to a method of Embodiment 5, further comprising converting a compound of the formula:

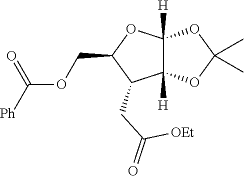 [SMILES: CCOC(=O)C[C@H]1[C@H]2OC(C)(C)O[C@H]2O[C@@H]1COC(=O)c1ccccc1]

to a compound of the formula:

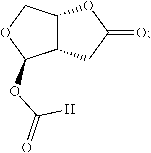 [SMILES: O=CO[C@H]1OC[C@@H]2OC(=O)C[C@H]12]

and converting said compound to a compound of the formula:

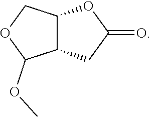

Embodiment 7 relates to a method of Embodiment 6, further comprising converting a compound of the formula:

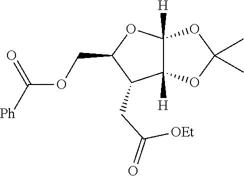 [SMILES: CCOC(=O)C[C@H]1[C@H]2OC(C)(C)O[C@H]2O[C@@H]1COC(=O)c1ccccc1]

to a compound of the formula:

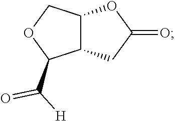 [SMILES: O=C[C@H]1OC[C@@H]2OC(=O)C[C@@H]21]

and converting said compound to a compound of the formula:

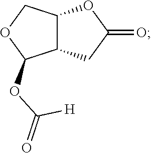 [SMILES: O=CO[C@H]1OC[C@@H]2OC(=O)C[C@H]12]

and converting said compound to a compound of the formula:

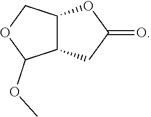

Embodiment 8 relates to a method of Embodiment 7, further comprising converting a compound of the formula:

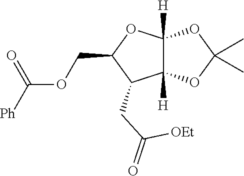 [SMILES: CCOC(=O)C[C@H]1[C@H]2OC(C)(C)O[C@H]2O[C@@H]1COC(=O)c1ccccc1]

to a compound of the formula:

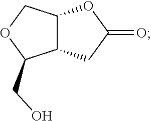 [SMILES: O=C1C[C@H]2[C@H](CO[C@@H]2CO)O1]

converting said compound to a compound of the formula:

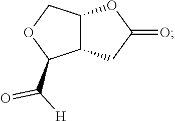 [SMILES: O=C[C@H]1OC[C@@H]2OC(=O)C[C@@H]21]

converting said compound to a compound of the formula:

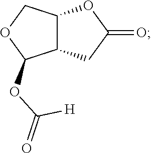 [SMILES: O=CO[C@H]1OC[C@@H]2OC(=O)C[C@H]12]

and converting said compound to a compound of the formula:

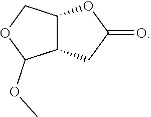

Embodiment 9 relates to a method of any preceding Embodiment, wherein the optically active P2-ligand precursor is compound of the formula:

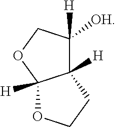

Embodiment 10 relates to a compound of formula

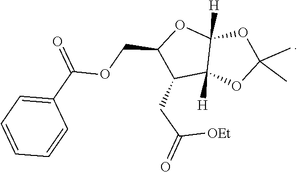 [SMILES: CCOC(=O)C[C@H]1[C@H]2OC(C)(C)O[C@H]2O[C@@H]1COC(=O)c1ccccc1]

Embodiment 11 relates to a compound of Embodiment 10, wherein the compound is enantiomerically pure.
Embodiment 12 relates to a compound of formula

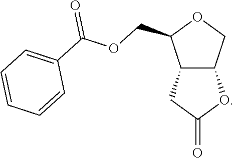 [SMILES: O=C1C[C@H]2[C@H](CO[C@@H]2COC(=O)c2ccccc2)O1]

Embodiment 13 relates to a compound of Embodiment 12, wherein the compound is enantiomerically pure.
Embodiment 14 relates to a compound of formula

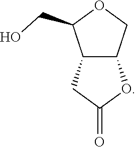 [SMILES: O=C1C[C@H]2[C@H](CO[C@@H]2CO)O1]

Embodiment 15 relates to a compound of Embodiment 14, wherein the compound is enantiomerically pure.
Embodiment 16 relates to an enantiomerically pure compound of formula

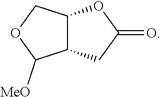 [SMILES: COC1OC[C@@H]2OC(=O)C[C@H]12]

Embodiment 17 relates to an enantiomerically pure compound of formula

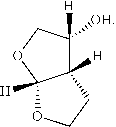

What is claimed is:

1. A method for making an optically active P2-ligand precursor comprising converting D-xylose or a derivative thereof or D-glucose or a derivative thereof to the optically active P2-ligand precursor.

2. The method of claim 1, wherein the D-xylose derivative is a compound of the formula:

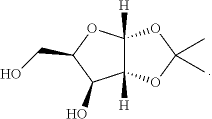

3. The method of claim 1, wherein the D-glucose derivative is a compound of the formula:

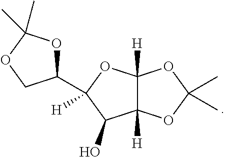

4. The method of any preceding claim, which comprises: converting D-xylose or a derivative thereof or D-glucose or a derivative thereof to a compound of the formula: 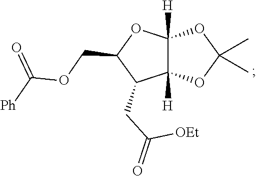 [SMILES: CCOC(=O)C[C@H]1[C@H]2OC(C)(C)O[C@H]2O[C@@H]1COC(=O)c1ccccc1]
and
converting said compound to the optically active P2-ligand precursor.

5. The method of claim 4, further comprising converting the compound of the formula: 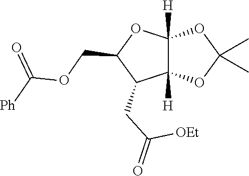 [SMILES: CCOC(=O)C[C@H]1[C@H]2OC(C)(C)O[C@H]2O[C@@H]1COC(=O)c1ccccc1]
to a compound of the formula: 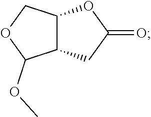
and then converting said compound to the optically active P2-ligand precursor.

6. The method of claim 5, further comprising converting a compound of the formula: 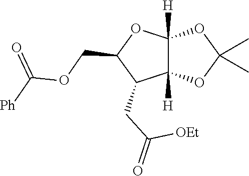 [SMILES: CCOC(=O)C[C@H]1[C@H]2OC(C)(C)O[C@H]2O[C@@H]1COC(=O)c1ccccc1]
to a compound of the formula: 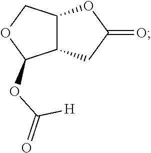 [SMILES: O=CO[C@H]1OC[C@@H]2OC(=O)C[C@H]12]
and then converting said compound to a compound of the formula:

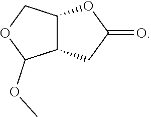

7. The method of claim 6, further comprising converting a compound of the formula: 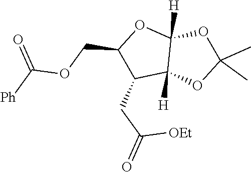 [SMILES: CCOC(=O)C[C@H]1[C@H]2OC(C)(C)O[C@H]2O[C@@H]1COC(=O)c1ccccc1]
to a compound of the formula: 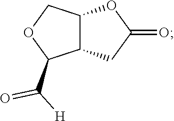 [SMILES: O=C[C@H]1OC[C@@H]2OC(=O)C[C@@H]21]
converting said compound to a compound of the formula: 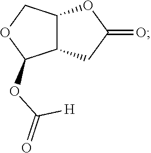 [SMILES: O=CO[C@H]1OC[C@@H]2OC(=O)C[C@H]12]
and then converting said compound to a compound of the formula:

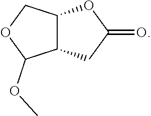

8. The method of claim 7, further comprising converting a compound of the formula: 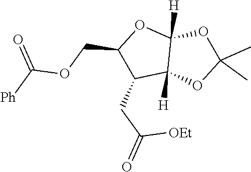 [SMILES: CCOC(=O)C[C@H]1[C@H]2OC(C)(C)O[C@H]2O[C@@H]1COC(=O)c1ccccc1]
to a compound of the formula: 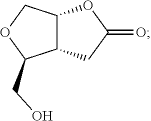 [SMILES: O=C1C[C@H]2[C@H](CO[C@@H]2CO)O1]
converting said compound to a compound of the formula: 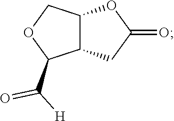 [SMILES: O=C[C@H]1OC[C@@H]2OC(=O)C[C@@H]21]
converting said compound to a compound of the formula: 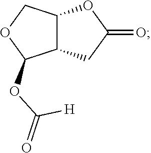 [SMILES: O=CO[C@H]1OC[C@@H]2OC(=O)C[C@H]12]
and then converting said compound to a compound of the formula:

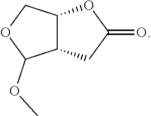

9. The method of any preceding claim, wherein the optically active P2-ligand precursor is compound of the formula:

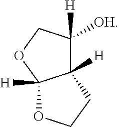

10. A compound of formula

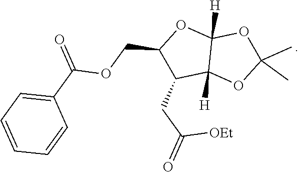 [SMILES: CCOC(=O)C[C@H]1[C@H]2OC(C)(C)O[C@H]2O[C@@H]1COC(=O)c1ccccc1]

11. The compound of claim 10, wherein the compound is enantiomerically pure.

12. A compound of formula

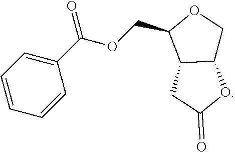 [SMILES: O=C1C[C@H]2[C@H](CO[C@@H]2COC(=O)c2ccccc2)O1]

13. The compound of claim 12, wherein the compound is enantiomerically pure.

14. A compound of formula

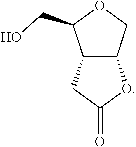 [SMILES: O=C1C[C@H]2[C@H](CO[C@@H]2CO)O1]

15. The compound of claim 14, wherein the compound is enantiomerically pure.

16. An enantiomerically pure compound of formula

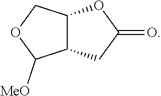 [SMILES: COC1OC[C@@H]2OC(=O)C[C@H]12]

17. An enantiomerically pure compound of formula [chemical structure]
